(19) United States
(12) Patent Application Publication
(10) Pub. No.: US 2022/0313717 A1
(43) Pub. Date: Oct. 6, 2022

(54) METHODS OF TREATING CRYPTOCOCCUS INFECTIONS

(71) Applicant: MATINAS BIOPHARMA NANOTECHNOLOGIES, INC., Bedminster, NJ (US)

(72) Inventors: Raphael J. MANNINO, Glen Gardner, NJ (US); Ruying LU, New Providence, NJ (US); Theresa MATKOVITS, West Orange, NJ (US)

(21) Appl. No.: 17/629,048

(22) PCT Filed: Aug. 13, 2020
(86) PCT No.: PCT/US2020/046114

§ 371 Date: Jan. 21, 2022

Related U.S. Application Data
(60) Provisional application No. 62/886,118, filed on Aug. 13, 2019.
(60) Provisional application No. 62/916,482, filed on Oct. 17, 2019.
(60) Provisional application No. 62/962,427, filed on Jan. 17, 2020.
(60) Provisional application No. 63/011,091, filed on Apr. 16, 2020.

(51) Int. Cl. A61K 31/7048; A61K 31/513; A61K 9/00; A61K 31/4196; A61K 9/127; A61K 47/18; A61K 47/24; A61K 47/22; A61K 47/38; A61K 47/14; A61K 47/02

(52) U.S. Cl. CPC A61K 31/7048; A61K 31/513; A61K 9/006; A61K 31/4196; A61K 9/1274; A61K 47/183; A61K 47/24; A61K 47/22; A61K 47/38; A61K 47/14; A61K 47/02

(57) ABSTRACT

The present disclosure is directed to a method of treating or preventing a fungal infection in a subject including a Cryptococcus spp., which method includes an induction treatment phase and a consolidation treatment phase, wherein the induction treatment phase includes: administering amphotericin B (cAMB) and 5-Flucytosine or an azole to the subject, wherein the cAMB and the 5-Flucytosine or the azole are mucosally administered. cAMB formulations are also disclosed.

### CROSS REFERENCE TO RELATED APPLICATIONS

This application claims the benefit of and relies on the filing date of U.S. Provisional Application No. 62/886,118, filed 13 Aug. 2019; U.S. Provisional Application No. 62/916,482, filed on 17 Oct. 2019; U.S. Provisional Application No. 62/962,427, filed on 17 Jan. 2020; and U.S. Provisional Application No. 63/011,091 filed on 16 Apr. 2020; the entire disclosure of each application is incorporated herein by reference.

### STATEMENT OF GOVERNMENT INTEREST

This invention was made with government support under grant number R01 NS110519 awarded by the National Institutes of Health. The U.S. Government has certain rights in this invention.

### FIELD

This application relates generally to methods of treating or preventing Cryptococcus spp. infections, particularly cryptococcal meningitis using, inter alia, mucosally administered formulations of amphotericin B. This application also relates to amphotericin B formulations for mucosal administration.

### BACKGROUND

Cryptococcosis is an opportunistic fungal infection causing an estimated 1 million cases and 625,000 deaths per year due to, e.g., cryptococcal central nervous system (CNS) infection. Early mortality from HIV-associated cryptococcal meningitis is particularly high, in large part due to the cost and toxicity of effective antifungals.

The ability of the antifungal, amphotericin B, to rapidly and consistently sterilize the cerebrospinal fluid (CSF) of cryptococcal patients suggests that this antifungal should be central to any cryptococcosis treatment strategy. However, the challenges associated with amphotericin B treatment often limits its use in resource-limited areas where cryptococcal meningitis is most prevalent. For example, amphotericin B is currently administered intravenously, which requires hospitalization, co-administration of IV fluids and supplemental electrolytes. Moreover, intravenously administered amphotericin B can be toxic, which necessitates rapid and reliable laboratory monitoring. Consequently, many treatment centers in low and middle income countries often find it difficult to use amphotericin B to treat cryptococcal infections. Accordingly, identifying more effective and less toxic antifungal therapy, which lessens dependence on costly, long courses of intravenous medications, associated with an increased risk of toxicity, is needed.

### SUMMARY

The present methods for treating or preventing cryptococcal infections provide surprising benefits in comparison to other methods for treating such diseases. For example, the present methods, which comprise the oral administration of antifungals, such as amphotericin B, are less toxic than those using intravenously administered antifungals, such as amphotericin B. Thus, the present methods may reduce or eliminate the need to administer antifungals, such as amphotericin B intravenously, and accordingly, eliminate the need in some instances for hospitalization and close monitoring of patients. Further, without the toxicities traditionally associated with amphotericin B, longer courses may be tolerated, which could decrease the incidence of relapse disease and immune reconstitution inflammatory syndrome (IRIS), a complication of antiretroviral therapy in patients with cryptococcosis. Moreover, the present methods may also be used in the absence of fluconazole, which provides a therapy for those subjects who have acquired sensitivity or resistance to fluconazole.

More particularly, the present disclosure is directed to a method of treating or preventing a fungal infection in a subject comprising a Cryptococcus spp., which method comprises an induction treatment phase and a consolidation treatment phase, wherein the induction treatment phase comprises: administering encochleated amphotericin B (cAMB) and 5-Flucytosine or an azole, typically 5-Flucytosine, to the subject, wherein the cAMB and the 5-Flucytosine or the azole are mucosally, e.g., orally or intransally, administered. The consolidation treatment phase can comprise orally administering encochleated amphotericin B to the subject in combination with an azole, such as fluconazole, to the subject.

The present disclosure also provides a composition comprising encochleated amphotericin B, wherein the encochleated amphotericin B comprises amphotericin B, a phospholipid, EDTA, water, vitamin E, calcium chloride, methylcellulose, methylparaben, proplyparaben, sodium hydroxide, dehydrated alcohol, monobasic potassium phosphate, potassium sorbate, acesulfame potassium and optionally flavoring.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIG. 1 is a schematic representation of a cochleate as described in the detailed description. The insert depicts the lipid strata of a cochleate, which contains a phospholipid bilayer (circles and tails), multivalent cation (unshaded circles) and an exemplified cargo moiety (hatched circles) protected within the cochleate.

FIG. 2 depicts a schematic of a macrophage engulfing a cochleate and its cargo. The insert depicts the opening of the cochleate and release of the cargo inside the macrophage as described in the detailed description.

FIG. 3 depicts a structural diagram of amphotericin B as described in the detailed description.

FIG. 4 is a schematic representation of an exemplary strategy for making amphotericin B-cochleates as described in the detailed description.

FIG. 5 depicts an exemplary preparation of geode cochleates as described in the detailed description.

FIG. 6 depicts a Phase I design for assessing the safety and tolerability of orally administered cAMB as described in the Examples.

FIG. 7 depicts a Phase II design for assessing the efficacy of orally administered cAMB as described in the Examples.

FIG. 8 depicts the mean cAMB plasma concentrations at various time points as described in the Examples.

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the disclosure and should not be interpreted as limiting the scope of the disclosure.

The present disclosure is directed a method of treating or preventing a fungal infection in a subject comprising a Cryptococcus spp., which method comprises an induction treatment phase and a consolidation treatment phase, wherein the induction treatment phase comprises: administering amphotericin B to the subject and administering 5-Flucytosine or an azole to the subject, wherein the amphotericin B comprises encochleated amphotericin B (cAMB), and wherein the cAMB and the 5-Flucytosine or the azole are mucosally, e.g., orally or intranasally, administered.

As used herein, the term “subject” refers to any animal including, without limitation, a human, a mouse, a rat, a rabbit, a non-human primate, or any other mammal. In one embodiment, the subject is a primate. Typically, the subject is a human.

The term “Cryptococcus spp.”, as used herein, refers to a species of fungi belonging to the genus Cryptococcus, which are basidiomycetes encapsulated yeasts. Such infections can be detected in a subject by any method known in the art, for example, by determining whether or not the subject is positive for a cryptococcal species antigen. Kits for performing such tests are commercially available, e.g., from IMMY Inc. (Norman, Okla.).

In some embodiments, the Cryptococcus spp. comprises Cryptococcus neoformans. C. neoformans was originally classified into serotypes A, B, C, D, and AD based on capsular agglutination reactions. More recently, C. neoformans has been divided into two varieties: C. neoformans var. grubii (formerly group A) and C. neoformans var. neoformans (formerly group D). C. neoformans is a ubiquitous pathogen found in most temperate regions of the world, but is commonly found in decaying organic matter and in many soil types, particularly soil types that have been enriched by animal and bird droppings.

In some embodiments, the Cryptococcus spp. comprises Cryptococcus gattii (formerly groups B and C). C. gattii can be divided into four molecular types including VGI, VGII, VGIII, and VGIV. Types VGII can be further divided into VGIIa, VGIIb, and VGIIc subtypes. C. gattii can be readily differentiated from C. neoformans by plating the isolate on canavanine-glycine-bromothymol (CGB) agar. CGB agar turns blue in the presence of this organism. C. gattii is traditionally found in tropical and subtropical geographic regions.

In some embodiments, the Cryptococcus spp. of the present disclosure, such as C. neoformans or C. gattii or, more typically, C. neoformans, infects an immunocompetent subject. As used herein, an immunocompetent subject is a subject, typically a mammalian subject, such as a human subject, who has the ability to produce a normal immune response following exposure to an antigen. More typically, however, the Cryptococcus spp. of the disclosure infects an immunocompromised subject. Immunocompromised subjects include subjects, typically mammalian subjects, such as human subjects, having HIV/AIDS, lymphoma, cirrhosis of the liver, or are organ transplant recipients, or subjects who are receiving immunosuppressive agents such as glucocorticosteroids, cytotoxic chemotherapy and/or TNF-α inhibitors.

In some embodiments, the fungal infection involves the skin, lung, prostate, bone and/or the central nervous system (CNS) of the subject. Typically, the present methods are used to treat infections of the CNS, such as cryptococcal meningeoencephalitis or cryptococcal meningitis.

Cryptococcal meningeoencephalitis and cryptococcal meningitis are invariably fatal without appropriate therapy; death may occur from 2 weeks to several years after symptom onset, typically 18 weeks. The most common symptoms of cryptococcal meningitis and meningoencephalitis are headache and altered mental status, including personality changes, confusion, lethargy and coma. Most typically, the present methods are used to treat or prevent cryptococcal meningitis.

### Induction

The methods of the disclosure, which are used to treat or prevent a Cryptococcus spp. infection, typically comprise an induction treatment phase. As used herein, “an induction treatment phase” refers to an initial phase of treatment, which reduces the fungal burden in the subject. Typically, the induction treatment phase is for a period of at least one week, such as at least two weeks, or such as at least four weeks or more. More typically, the induction period is for a period of one to four weeks, such as at least one to two weeks. Most typically, the induction period is for a period of two weeks.

In some embodiments, a reduction in fungal burden is determined by assessing the quantitative rate of fungal clearance from the cerebrospinal fluid (CSF). This quantitative rate of clearance, also known as early fungicidal activity (EFA), is generally logarithmic over the first weeks of treatment; thus, the clearance (EFA) can be estimated by linear regression of log₁₀ colony-forming units (CFU) per milliliter (mL) of CSF per day of therapy as a unit of measure (i.e. a change in log₁₀ CFU/mL CSF/day). Methods for assessing EFA are known in the art and described, for example, in Nussbaum et al., Clin. Infect. Dis. 2010; 50: 338-44, which is herein incorporated by reference in its entirety.

In some embodiments, the present induction treatment phase results in an EFA of at least −0.10 log₁₀ CFU/mL/day, such as at least −0.20 log₁₀ CFU/mL/day, such as at least −0.40 log₁₀ CFU/mL/day or such as at least −0.60 log₁₀ CFU/mL/day, typically at least −0.20 log₁₀ CFU/mL/day. In some embodiments, the EFA of the present induction phase ranges from −0.20 log₁₀ CFU/mL/day to −0.60 log₁₀ CFU/mL/day, such as from −0.20 log₁₀ CFU/mL/day to −0.29 log₁₀ CFU/mL/day.

Without being bound by theory, it is believed that there is an association between EFA during induction therapy and 10-week survival after a Cryptococcus spp. infection, such as a cryptococcal meningitis infection. The present methods, which may result in clearance values at a faster rate than −0.20 log₁₀ CFU/mL/day, also typically result in better survival after a Cryptococcus spp. infection, such as cryptococcal meningitis, in comparison to other treatment methods. However, once the EFA exceeds approximately −0.30 log₁₀ CFU/mL/day, there can be less obvious incremental gains in survival. For example, EFAs at a slower clearance rate than −0.20 log₁₀ CFU/ml CSF/day are typically associated with a higher 10-week mortality (>50%) while EFAs with a faster clearance rate than −0.30 log₁₀ CFU/ mL/day, are typically associated with similar mortality, i.e., 30-40%. Consequently, the present method, which may result in EFA values between −0.20 log₁₀ CFU/mL/day to −0.29 log₁₀ CFU/mL/day, can result in survival rates comparable to those methods that may result in faster fungal clearance rates.

In some embodiments, the induction treatment phase comprises mucosally, e.g., orally administering encochleated amphotericin B (cAMB) to a subject. Methods of encochleating compounds, including antifungal agents, such as amphotericin B, are known in the art and described herein.

In some embodiments, cAMB is administered to a subject in an amount ranging from about 7 mg/kg/day to about 29 mg/kg/day, such as about 14 mg/kg/day to about 29 mg/kg/day. Typically, the subject is a human subject receiving about 0.5 grams to about 2 grams cAMB per day in total, such as about 1 gram per day, such as about 1.5 grams per day, more typically about 2 grams per day. The daily dose of cAMB may be administered as a single dose or in divided doses over a 24 hour period. Typically, the cAMB is administered in divided doses, wherein each divided dose is ≤400 milligrams (mg), such as ≤375 mg, such as ≤300 mg, such as ≤333 mg, such as ≤286 mg, such as <250 mg, such as ≤200 mg, or such as ≤167 mg. For example, each divided dose of cAMB may range from about 150 mg to about 400 mg, such as about 167 mg to about 400 mg, such as about 167 mg to about 375 mg, or such as about 200 mg to about 300 mg.

In some particular embodiments during the induction treatment phase, a subject, such as a human subject, suffering from a Cryptococcus sp. infection, such as cryptococcus meningitis, is orally administered 4 divided doses of cAMB per day, each about 250 mg, every 4 hours to achieve a total daily cumulative dose of 1 gram over about a 12 hour period. In other embodiments, a subject, such as a human subject, is orally administered 5 divided doses of cAMB per day, each about 200 mg, every 3-4 hours to achieve a total daily cumulative dose of about 1 gram over about a 12-16 hour period. In some embodiments, a subject, such as a human subject, is orally administered 6 divided doses of cAMB per day, each about 167 mg, every 2-3 hours to achieve a total daily cumulative dose of about 1 gram over about a 10-15 hour period.

More typically during the induction treatment phase, a subject, such as a human subject, is orally administered 4 divided doses of cAMB per day, each about 375 mg, every 3-4 hours to achieve a total daily cumulative dose of 1.5 grams over about a 9-12 hour period. In other embodiments, a subject, such as a human subject, is orally administered 5 divided doses of cAMB per day, each about 300 mg, to achieve a total daily cumulative dose of about 1.5 gram over about a 16 hour period. In some embodiments, a subject, such as a human subject, is orally administered 6 divided doses of cAMB per day, each about 250 mg, every 2-3 hours to achieve a total daily cumulative dose of about 1.5 grams over about a 10-15 hour period.

Yet even more typically during the induction treatment phase, a subject, such as a human subject, is orally administered 5 divided doses of cAMB per day, each about 400 mg, every 3-4 hours to achieve a total daily cumulative dose of 2.0 grams, over about a 15-16 hour period. In other embodiments, a subject, such as a human subject, is orally administered 6 divided doses of cAMB per day, each about 333 mg, every 3.5 hours to achieve a total daily cumulative dose of about 2.0 gram over about a 17.5 hour period. In some embodiments, a subject, such as a human subject, is orally administered 7 divided doses of cAMB per day, each about 286 mg, every 3 hours to achieve a total daily cumulative dose of about 2.0 grams over about an 18 hour period.

Without being limited by theory, it is believed that the divided cAMB doses will be better tolerated by a subject than a single dose, e.g., result in less gastrointestinal side effects due to non-absorption of the cochleate carrier, for example, a phosphatidylserine cochleate carrier as described herein. Consequently, it is believed that the divided cAMB doses described herein will allow for higher cumulative daily dosing, such as up to 2 grams of cAMB per day, with decreased gastrointestinal adverse events in comparison to a single, undivided daily dose.

In some embodiments of the induction treatment phase, the multiple daily divided cAMB doses, typically a divided dose of ≤400 mg, which is administered to a subject as described herein, results in greater bioavailability than would be attained after administration of a single dose of cAMB, e.g., a single dose of 1, 1.5 or 2 grams of cAMB/day.

As used herein, bioavailability of a drug is defined as the proportion of a drug or other substance that enters the circulation when introduced into the body and so is able to have an active effect. As is well-known by an ordinary artisan, measures of bioavailability include the area under the plasma concentration-time curve (AUC), the concentration maximum (C_{maX}), and the time to Cmax (Tₘₐₓ). AUC is a measurement of the area under the plasma concentration-time curve, and is representative of the total drug exposure following administration of a single dose or multiple dose of a drug (Remington: The Science and Practice of Pharmacy, (Alfonso R. Gennaro ed. 2000), page 999). Cmaₓ is the maximum plasma concentration achieved after drug administration (Remington, page 999). Tₘₐₓ is the amount of time necessary to achieve the Cmaₓ after drug administration, and is related to the rate of absorption of a drug (Remington, page 999).

In some embodiments of the induction treatment phase, the multiple daily divided cAMB doses, typically a multiple divided dose of ≤400 mg as described herein, results in a higher AUC of drug exposure, such as encochleated amphotericin B drug exposure, than would be attained after administration of a single dose of the present cochleate composition, such as a single daily dose of cAMB, e.g., a single dose of 1, 1.5 or 2 grams of cAMB/day.

In some embodiments, the divided cAMB dose, typically a divided dose of ≤400 mg as described herein, is associated with less drug-related adverse events (AEs) or less severe drug-related AEs, than the number or severity of drug-related AEs associated with a single dose of cAMB.

As used herein, an adverse event or AE is any untoward medical occurrence in a subject due to administration of a pharmaceutical product. An AE may be classified as serious or non-serious. A serious adverse event is one that is fatal, life-threatening, requires re-hospitalization (e.g. after hospital discharge for the initial, pre-existing Cryptococcus infection, such as cryptococcal meningitis), results in persistent or significant disability or incapacity, results in congenital anomaly or birth defect, results in a medical event that may jeopardize the subject or may require immediate intervention to prevent one of the other foregoing outcomes (e.g. anaphylaxis). As used herein, a “non-serious” adverse event is any adverse event that does not meet the criteria of a serious adverse event.

Typically, AEs due to the oral administration of cAMB are gastrointestinal-related AEs. In some embodiments, the divided cAMB dose, typically a divided dose of ≥400 mg as described herein, is associated with less drug-related gastrointestinal AEs or less severe drug-related gastrointestinal AEs, than the number or severity of drug-related gastrointestinal adverse events associated with administration of a single daily cAMB dose, typically a single dose of 1 gram, 1.5 grams or 2 grams.

In some embodiments, mild gastrointestinal distress or nausea is associated with administration of a divided cAMB dose, wherein the mild gastrointestinal distress or nausea is a Grade 1 or a Grade 2 gastrointestinal or nausea event as described in Table 1. Typically, however, administration of the divided cAMB dose results in the absence of drug-related gastrointestinal distress and/or nausea of any Grade.

TABLE 1
Common Terminology Criteria for Adverse Events (CTCAE)¹

| Adverse | | | | |
|---|---|---|---|---|
| Event | Grade 1 | Grade 2 | Grade 3 | Grade 4 |
| Vomiting | 1-2 episodes^{a} | 3-5 episodes^{a} | >6 episodes^{a} | Vomiting |
| | per 24 hour | per 24 hour | per 24 hour | resulting in |
| | period | period | period | hypovolemic |
| | | | | shock^{b} |
| Diarrhea | <4 stools | 4 to 6 stools | >7 stools | Diarrhea |
| | per day | per day | per day | resulting in |
| | increased | increased | increased | hypovolemic |
| | from | from | from | shock^{b} |
| | baseline | baseline | baseline | |
| Nausea | Loss of | Reduced oral | Inadequate | |
| | appetite, | intake, | oral intake, | |
| | but no | but no | requiring | |
| | alteration | weight loss, | tube feeding, | |
| | in eating | dehydration, | intravenous | |
| | habits | or | feeding, or | |
| | | malnutrition | hospitalization | |

^{a}separated by > 5 minutes,
^{b}life threatening

In some embodiments during the induction treatment phase, a subject is orally administered a total daily cumulative dose of cAMB, each day for a period of 2 days of the induction treatment phase, more typically for a period of five days of the induction treatment phase, even more typically for a period of 9 days of the induction treatment phase, yet even more typically for a period of 10 days of the induction treatment phase, most typically for a period of 14 days of the induction treatment phase. In some embodiments, a subject is orally administered a total daily cumulative dose of cAMB on each day of the entire induction treatment phase. ¹ world wide web. eortc.be/services/doc/ctc/ctcae_4.03_2010-06-14_quickreference_5×7.pdf obtained 31 Aug. 2019, which is herein incorporated by reference in its entirety.

In some embodiments, the induction treatment phase further comprises administering intravenous amphotericin B (IV AMB) to the subject. However, without being bound by theory, it is believed that the oral administration of cAMB for any portion of the induction treatment phase may be used to decrease the number of days that intravenous administration of cAMB is needed for treatment of a cryptococcus infection, such as cryptococcal meningitis. For example, the administration of cAMB may allow for the absence of IV AMB administration or allow for a decrease in the number of days IV AMB is administered. For example, the IV AMB may only be administered for 10 days of the induction treatment phase, such as for only five days of the induction treatment phase.

The IV AMB may be administered simultaneously with the cAMB throughout the entirety of or for only a portion of the induction treatment phase. More typically, however, if IV AMB is administered, it is administered for only a portion of the inductive treatment phase and the cAMB is administered for only a portion of the inductive treatment phase. In some embodiments, the IV AMB and the cAMB are administered simultaneously only for a day or two of the induction treatment phase or are not administered simultaneously during the induction treatment phase. For example, cAMB and IV AMB may be administered simultaneously for at least one day of the induction treatment phase, such as at least two days of the induction treatment phase, e.g., simultaneous administration may be for one day of the induction treatment phase.

For example, in some embodiments, the induction treatment phase is 14 days in duration and comprises administering IV AMB from days 1-5 followed by administration of cAMB from days 5-14 of the induction period. In some embodiments, the induction treatment phase is 14 days in duration and comprises administering IV AMB from days 1-2 followed by administration of cAMB from days 2-14 of the induction treatment phase. In some embodiments, the induction treatment phase is 14 days in duration and comprises administering cAMB to a subject from days 1-5 of the induction treatment phase followed by administration of IV AMB from days 6-14 of the induction treatment phase. In some embodiments, the induction treatment phase is 14 days in duration and comprises administering IV AMB from days 1-2 followed by administration of cAMB from days 3-14 of the induction treatment phase. In some embodiments, the induction treatment phase is 14 days in duration and comprises administering cAMB to a subject from days 1-14 in the absence of IV AMB.

Typically, the IV AMB, when administered during the induction treatment phase, is in a dosage of from about 0.7 mg/kg/day to about 1.0 mg/kg/day. More typically, the IV AMB is administered to the subject during the induction treatment phase at a dosage of 1.0 mg/kg/day.

In some embodiments, the induction treatment phase further comprises orally administering 5-Flucytosine to the subject. 5-Flucytosine is a medication active against most strains of Candida and Cryptococcus. In some embodiments, the 5-Flucytosine is administered on each day of the induction treatment phase, e.g., days 1-14. In other embodiments, the 5-Flucytosine is administered during only part of the induction treatment phase, such as on days 7-14 of a 14-day induction treatment phase. Typically, however, 5-Flucytosine is administered on each day of the induction treatment phase. In some embodiments, 5-Flucytosine is orally administered during the induction treatment phase at a dosage of about 50 mg/kg/day to about 150 mg/kg/day, typically about 100 mg/kg/day.

In some embodiments, the 5-Flucytosine is encochleated. In some embodiments, the cochleate comprising amphotericin B of the present disclosure further comprises the 5-Flucytosine. Typically, however, the 5-Flucytosine is unencochleated.

In some embodiments, the induction treatment phase further comprises administering an azole to a subject in need thereof. In some embodiments, the azole is selected from fluconazole, ketoconazole, ravuconazole, albaconazole, itraconazole, posaconazole, isavuconazole and/or voriconazole. More typically, the azole is fluconazole. Fluconazole is a synthetic triazole antifungal agent, which is a highly selective inhibitor of fungal cytochrome P450 dependent enzymes. It is a potent CYP2C9 inhibitor and moderate CYP3A4 inhibitor.

In some embodiments, the azole, such as fluconazole, is administered during the induction treatment phase in the presence of 5-Flucytosine. Typically, the azole, such as fluconazole, if administered, is administered during the induction treatment phase in the absence of 5-Flucytosine.

In embodiments when an azole, such as fluconazole, is administered during the induction treatment phase, the azole, such as fluconazole, is administered on each day of induction. For example, the azole, such as fluconazole, may be administered on each of days 1-14 of a two week induction treatment phase. In other embodiments, the azole, such as fluconazole, is administered during only a part of the induction treatment phase, such as only on days 7-14 of a two week induction treatment phase. In some embodiments, the azole is fluconazole, which is orally administered during the induction treatment phase at a dosage ranging from about 200 mg/kg/day to about 1200 mg/kg/day, typically at a dosage of 800 mg/kg/day, more typically, at a dosage of 1200 mg/kg/day.

In some embodiments, the azole, such as fluconazole, is administered in divided doses. For example, a subject may receive 3 divided doses of 400 mg of fluconazole every three to four hours to achieve a daily cumulative dose of 1200 mg/kg/day of fluconazole.

In some embodiments, the azole, such as fluconazole, is encochleated. In some embodiments, the cochleate comprising amphotericin B of the present disclosure further comprises the azole, such as fluconazole. Typically, however, the azole, such as fluconazole, is unencochleated.

In the most typical embodiments, the induction treatment phase of the present disclosure includes the administration of cAMB in combination with 5-Flucytosine, particularly unencochleated 5-Flucytosine. The cAMB is, typically, orally administered daily at a total cumulative daily dosage of 1-2, grams, typically 2 grams on each day of the inductive treatment phase, typically 1-14 days. The cAMB is, typically, administered in combination with the oral administration of 5-Flucytosine, typically at a daily dosage of 100 mg/kg. The 5-Flucytosine is typically administered on each day of the induction treatment phase in combination with cAMB simultaneously or in series, in any order.

### Consolidation

In some embodiments, treatment is discontinued after the induction treatment phase of the present disclosure. In these embodiments, CSF cultures of the subject, after repeated lumbar punctures, are found to be negative and the subject exhibits substantial clinical improvement. However, more typically, the induction treatment phase of the present disclosure is followed by a consolidation treatment phase, also referred to as a “follow-up” treatment phase or an “eradication phase.” The length of the consolidation treatment phase may be for a period of about two to twelve weeks, more typically about 10 weeks.

In some embodiments, cAMB is orally administered during the consolidation treatment phase. In these embodiments, the cAMB is administered to a subject, such as a human subject, during the consolidation treatment phase in an amount ranging from about 7 mg/kg/day to about 29 mg/kg/day, such as about 14 mg/kg/day to about 29 mg/kg/day. Typically, during the consolidation treatment phase, the subject is a human subject receiving about 0.5 grams to about 2 grams cAMB per day in total, such as about 1 gram per day, such as about 1.5 grams per day, such as about 2 grams per day.

Typically, the cAMB total dosage/day during the consolidation treatment phase is less than the total dosage administered per day during the induction treatment phase. For example, the cAMB dosage is administered in an amount at least 0.5 grams less per day, such as at least 1.0 gram less per day, than the amount administered during the induction treatment phase. Typically, cAMB is administered to the subject, such as a human subject, during the consolidation treatment phase in an amount of about 1.5 grams/day, such as about 1.0 grams per day.

cAMB may be administered during the consolidation treatment phase as a single dose or in divided doses over a 24-hour period. Typically, cAMB is administered in divided doses, wherein each divided dose is ≤400 mg, such as ≤375 mg, such as ≤300 mg, such as ≤333 mg, such as ≤286 mg, such as <250 mg, such as ≤200 mg, or such as ≤167 mg. For example, each divided dose of cAMB may range from about 150 mg to about 400 mg, such as about 167 mg to about 400 mg, such as about 167 mg to about 375 mg, or such as about 200 mg to about 300 mg.

In some embodiments during the consolidation treatment phase, a subject suffering from a Cryptococcus sp. infection, such as cryptococcal meningitis, is orally administered 5 divided doses of cAMB per day, each about 400 mg, every 3-4 hours to achieve a total daily cumulative dose of about 2.0 grams over about a 15-16 hour period. In other embodiments, a subject, such as a human subject, is orally administered 6 divided doses of cAMB per day, each about 333 mg, every 3.5 hours to achieve a total daily cumulative dose of about 2.0 gram over about a 17.5 hour period. In some embodiments, a subject, such as a human subject, is orally administered 7 divided doses of cAMB per day, each about 286 mg, every 3 hours to achieve a total daily cumulative dose of about 2.0 grams over about an 18 hour period.

In some embodiments, during the consolidation treatment phase, a subject is orally administered 4 divided doses of cAMB per day, each about 375 mg, every 3-4 hours to achieve a total daily cumulative dose of about 1.5 grams over about a 9-12 hour period. In other embodiments, a subject, such as a human subject, is orally administered 5 divided doses of cAMB per day, each about 300 mg, to achieve a total daily cumulative dose of about 1.5 gram over about a 16 hour period. In some embodiments, a subject, such as a human subject, is orally administered 6 divided doses of cAMB per day, each about 250 mg every 2-3 hours to achieve a total daily cumulative dose of about 1.5 grams over about a 10-15 hour period.

Yet even more typically during the consolidation treatment phase, a subject is orally administered 4 divided doses of cAMB per day, each about 250 mg, every 4 hours to achieve a total daily cumulative dose of 1 about gram over about a 12 hour period. In other embodiments, a subject, such as a human subject, is orally administered 5 divided doses of cAMB per day, each about 200 mg every 3-4 hours to achieve a total daily cumulative dose of about 1 gram over about a 12-16 hour period. In some embodiments, a subject, such as a human subject, is orally administered 6 divided doses of cAMB per day, each about 167 mg, every 2-3 hours to achieve a total daily cumulative dose of about 1 gram over about a 10-15 hour period.

In some embodiments, a subject will receive a daily cAMB dose throughout the entirety of the consolidation treatment phase, e.g., each day of the consolidation treatment phase for a period of ten weeks. More typically, however, cAMB is administered for only a portion of the consolidation treatment phase, e.g., daily for a period of at least one week, such as at least two weeks, such as at least three weeks, such as at least four weeks, such as at least eight weeks. Typically, cAMB is administered during the consolidation treatment phase for one to four weeks, such as two to four weeks, most typically four weeks.

In some embodiments, the consolidation treatment phase further comprises orally administering an azole in addition to cAMB. In some embodiments, the azole is selected from fluconazole, ketoconazole, ravuconazole, albaconazole, itraconazole, posaconazole, isavuconazole and/or voriconazole. In some embodiments, the azole is encochleated. In some embodiments, the cochleate comprising the amphotericin B of the present disclosure further comprises an azole. Typically, however, the azole is unencochleated. More typically, the azole is unencochleated fluconazole.

In some embodiments, the azole, such as fluconazole, is administered on all days of the consolidation treatment phase. In other embodiments, the azole, such as fluconazole, is administered only during a portion of the consolidation treatment phase, such for at least two weeks, such as for at least four weeks, such as at least eight weeks, such as at least 10 weeks, such as between two weeks and ten weeks, such as between six weeks and ten weeks. Typically, the azole, such as fluconazole, is administered daily for the entire consolidation treatment phase, most typically for ten weeks.

In some embodiments, the azole is fluconazole, which is orally administered during the consolidation treatment phase at a dosage ranging from about 400 mg/kg/day to about 800 mg/kg/day. Typically, fluconazole is administered during the consolidation treatment phase at a dosage of about 800 mg/kg/day. In some embodiments, the azole, such as fluconazole, is administered during the consolidation treatment phrase in divided doses. For example, a subject, such as a human subject, may receive 2 divided doses of 400 mg/kg/day of orally administered fluconazole every three to six hours to achieve a total daily cumulative dose of 800 mg/kg/day of fluconazole.

### Maintenance

In some embodiments, after completion of the induction treatment phase and an optional consolidation treatment phase, the method of the present disclosure may further, optionally, comprise a maintenance phase. In some embodiments, the maintenance phase prevents recurrence of the Cryptococcus sp. infection, such as cryptococcus meningitis.

In some embodiments, the maintenance phase comprises the daily oral administration of an antifungal drug to a subject for a period of six months to two years, typically one year.

In some embodiments, the antifungal drug administered during the maintenance phase is an azole, which is orally administered. In certain embodiments, the azole is encochleated. More typically, however, the azole is unencochleated.

In some embodiments, the azole is selected from fluconazole, ketoconazole, ravuconazole, albaconazole, itraconazole, posaconazole, isavuconazole and/or voriconazole. More typically, the azole is fluconazole. Typically, the fluconazole is orally administered during the maintenance phrase, e.g. in dosages ranging from about 200 mg/kg/day to about 400 mg/kg/day. More typically, the fluconazole is orally administered during the maintenance phrase, e.g. in dosages of about 200 mg/kg/day.

In some embodiments, during the induction, consolidation and/or maintenance phase additional anti-fungal compounds may be administered. The additional antifungal compounds may be encochleated or unencochleated. The additional anti-fungal compounds contemplated for administration during the induction, consolidation and/or maintenance phases of the present methods include those capable of inhibiting the synthesis of a cell wall component, such as glycosylphosphatidylinositol (GPI)-anchored mannoproteins, e.g., E1210, an orally active molecule, which has in vitro activity against Cryptococcus spp. In other embodiments, the additional anti-fungal compound is an ergosterol synthesis inhibitor, such as VT-1129, which is orally available, shows good CNS penetration and is fungicidal in mouse models of Cryptococcus spp. infection. Other compounds that may be administered during the induction, consolidation and/or maintenance phase include sertraline. Biologics such as TNF-γ are also contemplated for administration during the induction, consolidation and/or maintenance phase of the present disclosure.

### Cochleates

As disclosed herein, the antifungal compounds of the present method, such as amphotoricin B, 5-Flucytosine and/or an azole, such as flucanazole, may be encochleated. Cochleates are anhydrous, stable, multi-layered lipid crystals which spontaneously form upon the interaction of negatively charged lipids, such as phosphatidylserine, and divalent cations, such as, calcium (see, for example, U.S. Pat. Nos. 4,078,052; 5,643,574; 5,840,707; 5,994,318; 6,153,217; 6,592,894, as well as PCT Publ. Nos. WO 2004/091572; WO 2004/091578; WO 2005/110361, WO 2012/151517 and WO2014/022414, U.S. Patent Publication No. 2014/220108 and Patent Publication No. 2010/0178325; each of which is incorporated fully herein by this reference). Typically, these are referred to as crystal cochleates. A variation of the crystal cochleate is known as the geode cochleate, or a geodate, as described, for example, in U.S. Pat. Publ. 2013/0224284, the entire disclosure of which is incorporated herein by reference.

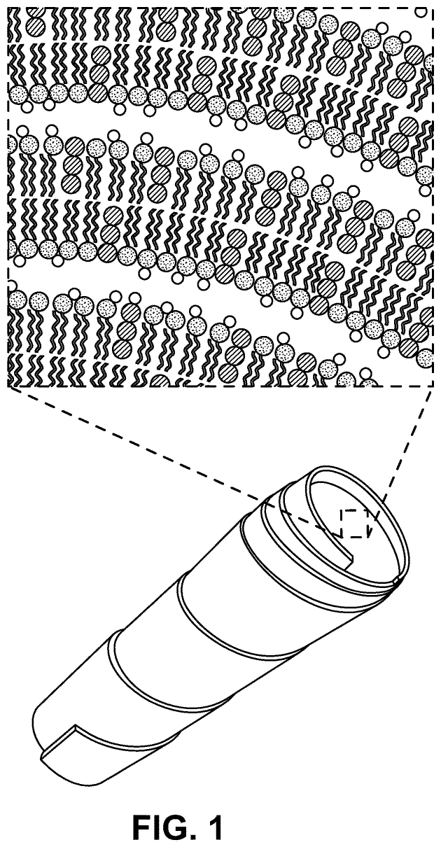

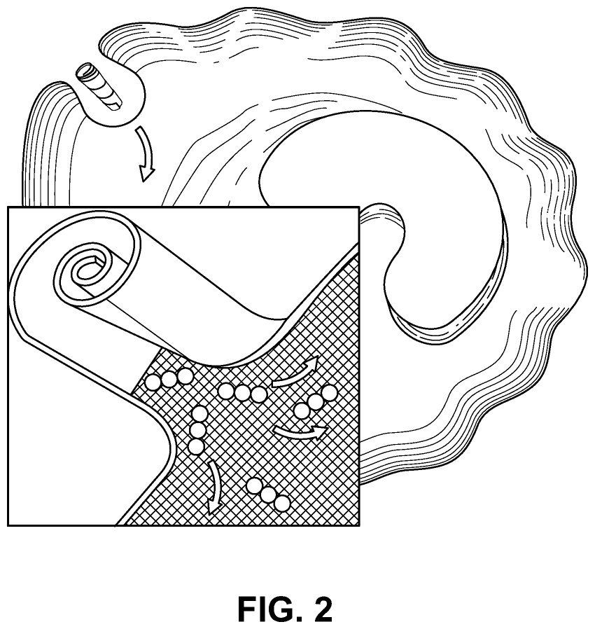

Cochleates have a unique multilayered structure consisting of a large, continuous, solid, phospholipid bilayer sheet or strata rolled up in a spiral or as stacked sheets, with no internal aqueous space (FIG. 1). This unique structure provides protection from degradation for associated “encochleated” molecules. Since the entire cochleate structure is a series of solid layers, components within the interior of the cochleate structure remain intact, even though the outer layers of the cochleate may be exposed to harsh environmental conditions or enzymes. Divalent cation concentrations in vivo in serum and mucosal secretions are such that the cochleate structure is maintained. Hence, the majority of cochleate-associated molecules are present in the inner layers of a solid, stable, impermeable structure. Once within the interior of a cell, however, the low calcium concentration results in the opening of the cochleate crystal and release of the molecule that had been formulated into cochleates (FIG. 2). Accordingly, cochleate formulations remain intact in physiological fluids, including mucosal secretions, plasma and gastrointestinal fluid, thereby mediating the delivery of biologically active compounds by many routes of administration, including mucosal, e.g., oral or intransal administration.

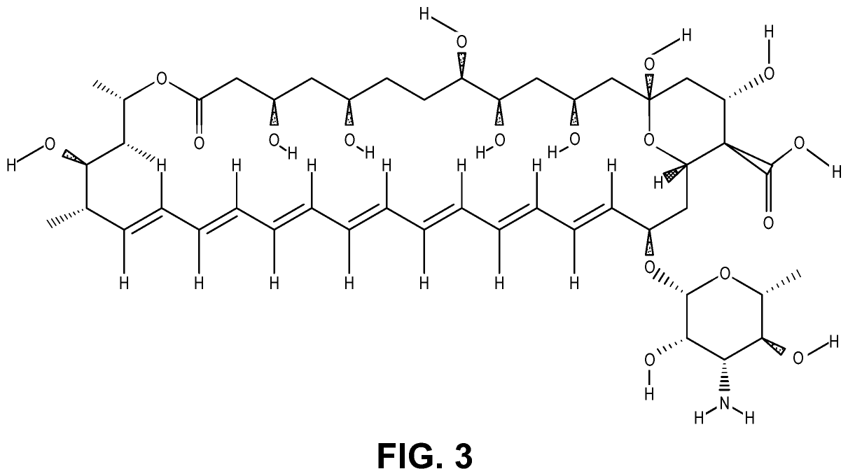

Typical cochleate structures include a lipid strata comprising alternating divalent cations and phospholipid bilayers that include at least one negatively charged phospholipid. Typically, a cargo moiety, such as an antifungal agent, for example, amphotericin B (FIG. 3) is sequestered within the lipid strata of the cochleate.

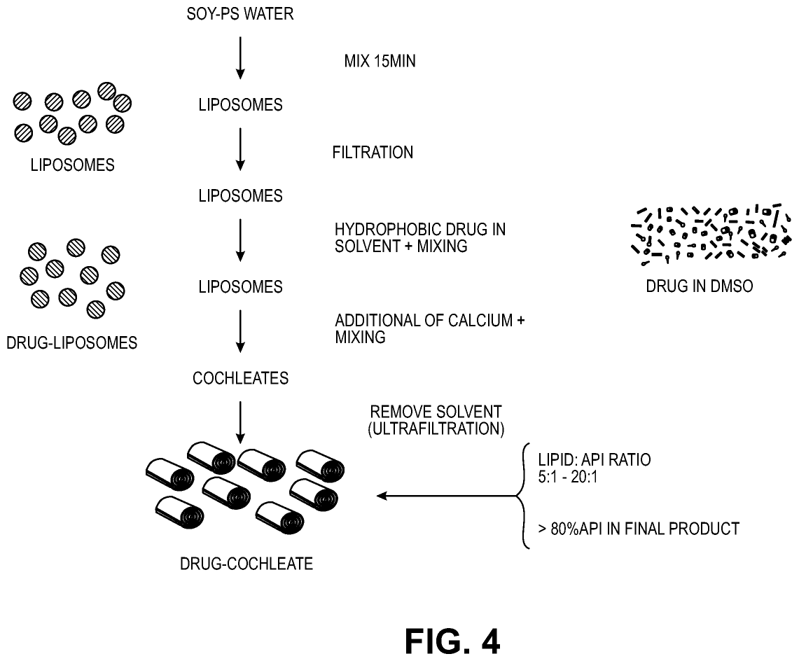

Cochleates can be made using known methods. In one typical implementation, the method described in U.S. Patent Publication No. 2014/220108 is used to make the cochleates of the present disclosure, which is herein incorporated by reference in its entirety. A summary of this process is shown in FIG. 4. In this method, a hydrophobic antifungal compound, such as amphotericin B, is typically dissolved in solvent, e.g., dimethylsulfoxide and filtered through e.g., a 0.22 μm filter and combined with e.g., 2000 milligrams 50% soy phosphatidylserine (PS) liposomes in 200 milliliters sterile water (the PS liposomes are first filtered through e.g., 5, 0.8, and 0.45 μm filters) to form liposomes containing the antifungal, such as AmB. To the resultant mixture a cation, such as a multivalent or divalent cation, can be added. The addition of a multivalent or divalent cation results in the collapse of the liposomes, and the formation of the sheets of cation-chelated phospholipid bilayers, which roll up or stack to form cochleates containing antifungal, such as amphotericin B. The antifungal-containing cochleates, such as amphotericin B-containing cochleates, may be dried under lyophilization. Sterile water may be added to the dried powder, anti-fungal cochleates to prepare a suspension. The suspension may be stored at 4° C. in the absence of light.

Other methods for making cochleates containing antifungals include the trapping-high pH method, the trapping-film method and the hydrogel method. In the trapping-high pH method, lipid powder and an antifungal compound, e.g., amphotericin B, are mixed in a lipid/antifungal molar ratio of e.g., 10:1 in an e.g., sterile polypropylene tube. Buffer, e.g., TES [N-Tris(hydroxymethyl)-methyl-2-aminomethane sulfonic acid] (pH 7.4) is added. Multilamellar liposomes are formed after vortexing. The pH is then increased to, e.g., 11.5, by the addition of e.g., 1 N NaOH, to solubilize the antifungal compound, e.g., amphotericin B. The absence of amphotericin B crystals and the presence of liposomes may be monitored by using phase contrast and polarization optical microscopy. Multivalent or divalent cation, such as calcium chloride, is added slowly to the antifungal liposome suspension at a lipid/cation molar ratio of e.g., 2:1, to form the cochleates. The external pH may then be adjusted to pH 7.

In the trapping film method, the antifungal compound, e.g., amphotericin B, is dissolved in solvent, e.g., methanol, with brief sonication and the solution is added to lipids in chloroform. The antifungal, e.g., amphotericin B, is readily soluble in the chloroform/methanol mixture. The mixture may then be dried to a film using a rotary evaporator and gently warmed at e.g., 35° C.-40° C., under reduced pressure (1 bar). The dried lipid film may then be hydrated with deionized water and sonicated. The antifungal-liposome size should be around 50 nanometers. To form the cochleates, a multivalent or divalent cation solution, e.g., calcium chloride in solution, is slowly added to the liposome suspension to form the cochleates.

To prepare antifungal cochleates using the hydrogel method, an antifungal compound, such as amphotericin B, is dissolved in methanol and added to lipids in chloroform at, e.g., a 10:1 molar ratio, and the mixture is then dried into a drug-lipid film using a rotary evaporator. The film may then be hydrated with deionized water and the drug-lipid suspension sonicated until small liposomes containing the anti-fungal compounds are obtained. The antifungal-liposome suspension may then be mixed with e.g., 40% w/w dextran-500,000 in a suspension of, e.g., 2/1 v/v dextran/liposome. This mixture is then injected using a syringe into e.g., 15% w/w PEG-8000 under magnetic stirring (800-1000 rpm). An aqueous-aqueous emulsion of antifungal liposomes/dextran droplets dispersed in a PEG continuous phase is obtained. A multivalent or divalent cation solution, e.g., calcium chloride in solution, is then added to the emulsion. Stirring is continued to allow for the slow formation of small-sized antifungal cochleates, which are sequestered in the dextran droplets. The polymer is then washed by the addition of a washing buffer containing e.g., 1 mM CaCl₂ and 150 mM NaCl.

As recognized by an ordinary artisan, many parameters, including pH, salt concentration, agitation method and rate, cation type, concentration, and rate of addition, lipid composition, concentration, and ratio of lipid to other material, etc., affect the formulation, and can be varied in order to optimize the encochleation of a particular material.

In some embodiments, a hydrophilic anti-fungal compound, such as 5-Flucytosine or an antifungal compound containing a hydrophilic domain, such as fluconazole, may also be formulated into a cochleate. Methods for incorporating such compounds into cochleates are well known in the art and are described, for example, in U.S. Patent Publication No. 2014/220108. Without wishing to be bound to any particular theory, it is believed that hydrophilic molecules or large molecules with hydrophilic domains, such as active pharmaceutical ingredients (APIs) of interest including the antifungal compounds of the present disclosure, can be formulated into cochleates in an enhanced manner by associating the API with a lipid domain that acts like a “raft”, and which remains intact and imbedded within the cochleate crystal matrix. Such lipids include “neutral lipids” as known in the art and described herein.

In a typical implementation, the multivalent cation described herein, which may be used to collapse the liposomes into cochleates, is a divalent metal cation, such as calcium, zinc, magnesium, and barium. In a more typical implementation, the divalent metal cation is calcium.

In some implementations, the ratio of anti-fungal agent to lipid (wt/wt) ranges from 1:1 to 1:50, or any range in between, such as, 1:2, 1:3, 1:4, 1:6, 1:8, 1:10, 1:12, 1:15, 1:20 and 1:25, typically 1:1 to 1-1:20, such as 1:2.5 to 1:10, typically 1:10.

The liposome used during the formation of the cochleates may be multilamellar (MLV) or unilamellar (ULV), including small unilamellar vesicles (SUV). The concentration of lipid in these liposomal solutions can be from about 0.1 mg/ml to 500 mg/ml. Typically, the concentration of lipid is from about 0.5 mg/ml to about 50 mg/ml, more typically from about 1 mg/ml to about 25 mg/ml.

A size-regulating agent may be introduced during the method of making the cochleate. A size-regulating agent, as used herein, refers to an agent that reduces the particle size of a cochleate. As used herein, the term “particle size” refers to the particle diameter, or in case the particles are not spherical, to the largest extension in one direction of the particle. The particle size of cochleates can be measured using conventional methods, such as a submicron particle size analyzer. In certain embodiments, the size regulating agent is a lipid-anchored polynucleotide, a lipid-anchored sugar (glycolipid), or a lipid-anchored polypeptide. In other embodiments, the size regulating agent is a bile salt, such as oxycholate, cholate, chenodeoxycholate, taurocholate, glycocholate, taurochenodeoxycholate, glycochenodeoxycholate, deoxycholate, or lithocholate. Bile salts are bile acids compounded with a cation, usually sodium. Bile acids are steroid acids found predominantly in the bile of mammals and are commercially available.

In certain embodiments, the size-regulating agent is added to the lipid or liposomes before formation of the precipitated cochleate. For example, in one embodiment, the size-regulating agent is introduced into a liposomal suspension from which cochleates will subsequently be formed (e.g., by addition of cation or dialysis). Alternatively, the size-regulating agent may be introduced to a lipid solution, before or after addition of a pharmacologically active agent.

In some embodiments, the cochleates of the present disclosure can optionally include one or more aggregation inhibitors. The term “aggregation inhibitor,” as used herein, refers to an agent that inhibits aggregation of cochleates. The aggregation inhibitor typically is present at least on the surface of the cochleate, and may only be present on the surface of the cochleate (e.g., when the aggregation inhibitor is introduced after cochleate formation). Aggregation inhibitors can be added before, after, or during cochleate formation. A person of ordinary skill in the art will readily be able to determine the amount of aggregation inhibitor needed to form cochleates of the desired size with no more than routine experimentation.

Suitable aggregation inhibitors that can be used in accordance with the present disclosure, include but are not limited to at least one of the following: casein, kappa-casein, milk, albumin, serum albumin, bovine serum albumin, rabbit serum albumin, methylcellulose, ethylcellulose, propylcellulose, hydroxycellulose, hydroxymethyl cellulose, hydroxyethyl cellulose, hydroxypropyl cellulose, hydroxypropylmethyl cellulose, polyvinyl pyrrolidone, carboxymethyl cellulose, carboxyethyl cellulose, pullulan, polyvinyl alcohol, sodium alginate, polyethylene glycol, polyethylene oxide, xanthan gum, tragacanth gum, guar gum, acacia gum, arabic gum, polyacrylic acid, methylmethacrylate copolymer, carboxy polymer, amylose, high amylose starch, hydroxypropylated high amylose starch, dextrin, pectin, chitin, chitosan, levan, elsinan, collagen, gelatin, zein, gluten, carrageenan, carnauba wax, shellac, latex polymers, milk protein isolate, soy protein isolate, whey protein isolate and mixtures thereof.

Any suitable lipid can be used to make the cochleate. In one embodiment, the lipid includes one or more negatively charged lipids. As used herein, the term “negatively charged lipid” includes lipids having a head group bearing a formal negative charge in aqueous solution at an acidic, basic or physiological pH, and also includes lipids having a zwitterionic head group. In one embodiment, the negatively charged lipid is a phospholipid.

The cochleates can also include non-negatively charged lipids (e.g., positive and/or neutral lipids). Typically, the cochleates include a significant amount of negatively charged lipids. In certain embodiments, a majority of the lipid is negatively charged. In one embodiment, the lipid is a mixture of lipids, comprising at least 50% negatively charged lipid, such as a phospholipid. In another embodiment, the lipid includes at least 75% negatively charged lipid, such as a phospholipid. In other embodiments, the lipid includes at least 85%, 90%, 95% or 98% negatively charged lipid, such as a phospholipid. In yet other embodiments, the negatively charged lipid (e.g., phospholipid) comprises between 30%-70%, 35%-70%, 40%-70%, 45%-65%, 45%-70%, 40%-60%, 50%-60%, 45%-55%, 45%-65%, or 45%-50% of the total lipid in the cochleate. In certain embodiments, the negatively charged lipid (e.g., phospholipid) comprises between 40%-60% or 45%-55% of the total lipid in the cochleate. In some embodiments, the negatively charged lipid (e.g., phospholipid) comprises between 30%-70%, 35%-70%, 40%-70%, 45%-65%, 45%-70%, 40%-60%, 50%-60%, 45%-55%, 45%-65%, or 45%-50% of the total lipid in the non-hydrophobic domain component of the cochleate. In certain embodiments, the negatively charged lipid (e.g., phospholipid) comprises between 40%-60% or 45%-55% of the total lipid in the non-hydrophobic domain component of the cochleate. In some embodiments, the negatively charged lipid is a phospholipid and comprises between 30%-70%, 35%-70%, 40%-70%, 45%-65%, 45%-70%, 40%-60%, 50%-60%, 45%-55%, 45%-65%, or 45%-50% of the total phospholipid in the cochleate or in the non-hydrophobic domain component of the cochleate. In some embodiments, the negatively charged lipid is a phospholipid and comprises between 40%-60% or 45%-55% of the total phospholipid in the cochleate or in the non-hydrophobic domain component of the cochleate.

The negatively charged lipid can include egg-based lipids, bovine-based lipids, porcine-based lipids, plant-based lipids or similar lipids derived from other sources, including synthetically produced lipids. The negatively charged lipid can include phosphatidylserine (PS), dioleoylphosphatidylserine (DOPS), phosphatidic acid (PA), phosphatidylinositol (PI), and/or phosphatidyl glycerol (PG) and or a mixture of one or more of these lipids with other lipids. Additionally or alternatively, the lipid can include phosphatidylcholine (PC), phosphatidylethanolamine (PE), diphosphotidylglycerol (DPG), dioleoyl phosphatidic acid (DOPA), distearoyl phosphatidylserine (DSPS), dimyristoyl phosphatidylserine (DMPS), dipalmitoyl phosphatidylglycerol (DPPG) and the like. In another embodiment, the phosphatidylserine is egg or bovine derived phosphatidylserine.

### Cochleates Containing Soy Lipids

In some typical embodiments, the cochleates, including the geode cochleates, described herein below, are prepared using legume-based phospholipids, more typically soy-based lipids. Such soy-based lipids can be natural or synthetic. Even more typically, the soy-based lipids are soy phospholipids, such as soy phosphatidylserine is in an amount of 40%-74% by weight of the lipid component of the cochleates. Alternatively, the soy phosphatidylserine can be about 40%, 45%, 50%, 55%, 60%, 65% or 70% or any incremental value thereof, by weight of the lipid component of the cochleates. It is to be understood that all values, and ranges between these values and ranges are meant to be encompassed by the present disclosure. In a typical embodiment, the phospholipid comprises 45-70% soy phosphatidylserine. In a more typical embodiment, the phospholipid comprises 45-55% soy phosphatidylserine.

Soy phosphatidylserine is commercially available, e.g., from Avanti Polar Lipids, Inc. Alabaster, Ala. Alternatively, soy phosphtidylserine can be purified from soy phospholipid compositions, which are mixtures of several soy phospholipids, according to well-known and standard purification techniques.

In some embodiments, neutral lipids are used in combination with the soy phosphatidylserine to make the instant cochleates. As used herein, the term “neutral lipids” include any of a number of lipid species, which exist either in an uncharged or neutral zwitterionic form at physiological pH and, thus, are included within the group of lipids lacking an anionic function. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the cochleate compositions described herein is generally guided by consideration of, e.g., cochleate size and stability. Lipids having a variety of acyl chain groups of varying chain length and degree of saturation are available or may be isolated or synthesized by well-known techniques. In one group of embodiments, lipids containing saturated fatty acids with carbon chain lengths in the range of C₁₄ to C₂₂ can be used. In another group of embodiments, lipids with mono or di-unsaturated fatty acids with carbon chain lengths in the range of C₁₄ to C₂₂ can be used. In yet another group of embodiments, lipids with mono or di-unsaturated fatty acids with carbon chain lengths in the range of C₈ to C₁₂ can be used. Additionally, lipids having mixtures of saturated and unsaturated fatty acid chains can be used.

In some embodiments, the neutral lipids used in the present disclosure are DOPE, DSPC, DPPC, POPC, or any related phosphatidylcholine. The neutral lipids useful in the present disclosure may also be composed of sphingomyelin, dihydrosphingomyeline, or phospholipids with other head groups, such as serine and inositol.

In a typical implementation, 99.9% pure dioleoyl phosphatidylserine, 99.9% pure soy phosphatidylserine, 75% soy phosphatidylserine and 50% soy phosphatidylserine, are used to manufacture cochleates. The lipid composition of 99.9% pure phosphatidylserine is typically modified by the addition of neutral lipids, including, but not limited to sphingomyelin and/or phosphatidylcholine. When lower purity phosphatidylserine (e.g., 50% soy phosphatidylserine) is used as a starting material, the lower purity phosphatidylserine can be subjected to extraction steps to remove unwanted impurities, such as, nucleases.

### Geode Cochleates

In some implementations, a cochleate of the present disclosure is a geode cochleate, or a geodate, as described, for example, in U.S. Patent Publication 2013/0224284, the entire disclosure of which is incorporated herein by reference. Geode cochleates further comprise a lipid monolayer comprising a negatively charged phospholipid, where the lipid monolayer surrounds a hydrophobic domain, such as an oil, and a cargo moiety, such as an antifungal compound as described herein, which is dispersed within the hydrophobic domain. The lipid monolayer is sequestered within the lipid strata of the geode cochleate.

As used herein, a “hydrophobic domain” is a composition that is sufficiently hydrophobic in nature to allow formation of a lipid monolayer about its periphery. A hydrophobic domain typically includes a hydrophobic composition, such as oil or fat, associated with a cargo moiety, such as an antifungal compound of the disclosure, such as amphotericin B. In certain embodiments, the ratio between the hydrophobic domain (HD) and the phospholipid component of the geode cochleate (PPLGD) HD:PPLGD or the castor oil domain (COD) and phospholipid component of the geode cochleate (PPLGD) COD:PPLGD is 1:20 or less, 1:15 or less, 1:10 or less, 1:8 or less, 1:6 or less, 1:5 or less, 1:4 or less, 1:3.5 or less, 1:3 or less, 1:2.75 or less, 1:2.5 or less, 1:2.25 or less, 1:2 or less, 1:1.75 or less, 1:1.5 or less, 1:1.25 or less 1:1 or less.

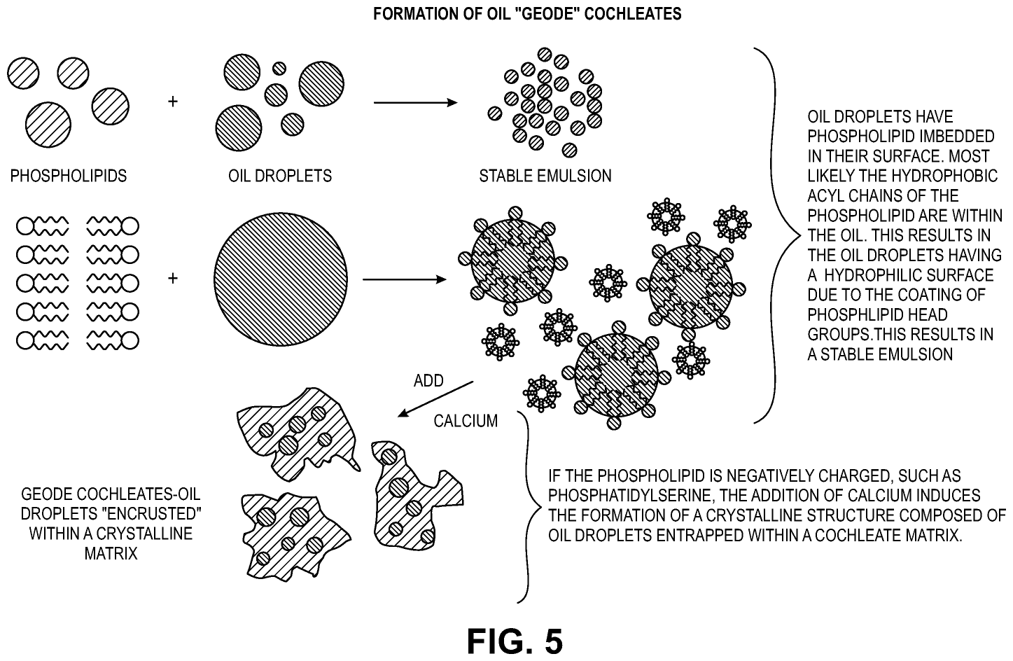

FIG. 5 shows an exemplary schematic of how geode cochleates can be made. In this exemplary method, a phospholipid (represented as an open ring) is combined with a hydrophobic domain (shaded circles), such as an oil, and mixed to form a stable emulsion comprising liposomes and lipid monolayers surrounding the hydrophobic domain. A cargo moiety, such as an antifungal compound, etc., may be dispersed within the hydrophobic domain. The hydrophobic domains have phospholipids imbedded in their surface. Without intending to be bound by any theory, it is believed that the hydrophobic acyl chains of the phospholipid are within the hydrophobic domains, resulting in the hydrophobic domains having a hydrophilic surface due to the coating of the phospholipid head groups and forming a stable emulsion. If the phospholipid is negatively charged, such as with phosphatidylserine, the addition of a divalent cation, such as calcium, induces the formation of a crystalline structure (or lipid strata) comprising alternating divalent cations and phospholipid bilayers. The lipid strata are represented with hatching. In a geode cochleate, the lipid monolayers surrounding the hydrophobic domain are “encrusted” or “entrapped” within the crystalline matrix, akin to a “geode.”

### Cochleate Formulations

The cochleates as described herein for use in the present methods comprise pharmaceutical compositions. Suitable preparation forms for the pharmaceutical compositions disclosed herein include, for example, tablets, capsules, soft capsules, granules, powders, suspensions, emulsions, microemulsions, nanoemulsions, unit dosage forms, rings, films, suppositories, solutions, creams, syrups, transdermal patches, ointments and gels. Typically, however, the cochleates are prepared for mucosal, e.g., oral or intranasal, typically oral administration.

The pharmaceutical compositions can include other pharmaceutically acceptable excipients, such as a buffer (e.g., Tris-HCl, acetate, phosphate) of various pH and ionic strength; an additive such as albumin or gelatin to prevent absorption to surfaces; a protease inhibitor; a permeation enhancer; a solubilizing agent (e.g., glycerol, polyethylene glycerol); an anti-oxidant (e.g., ascorbic acid, sodium metabisulfite, butylated hydroxyanisole); a stabilizer (e.g., hydroxypropyl cellulose, hydroxypropylmethyl cellulose); a viscosity increasing agent (e.g., carbomer, colloidal silicon dioxide, ethyl cellulose, guar gum); a sweetener (e.g. aspartame, citric acid); a preservative (e.g., Thimerosal, benzyl alcohol, parabens); a flow-aid (e.g., colloidal silicon dioxide), a plasticizer (e.g., diethyl phthalate, triethyl citrate); an emulsifier (e.g., carbomer, hydroxypropyl cellulose, sodium lauryl sulfate); a polymer coating (e.g., poloxamers or poloxamines, hypromellose acetate succinate); a coating and film forming agent (e.g., ethyl cellulose, acrylates, polymethacrylates, hypromellose acetate succinate); an adjuvant; a pharmaceutically acceptable carrier for liquid formulations, such as an aqueous (water, alcoholic/aqueous solution, emulsion or suspension, including saline and buffered media) or non-aqueous (e.g., propylene glycol, polyethylene glycol, and injectable organic esters such as ethyl oleate) solution, suspension, emulsion or oil; and a parenteral vehicle (for subcutaneous, intravenous, intraarterial, intrathecal or intramuscular injection), including but not limited to, sodium chloride solution, Ringer's dextrose, dextrose and sodium chloride, lactated Ringer's and fixed oils.

In certain embodiments, the pharmaceutical composition comprises a salt, such as NaCl or a bile salt, such as oxycholate, cholate, chenodeoxycholate, taurocholate, glycocholate, taurochenodeoxycholate, glycochenodeoxycholate, deoxycholate or lithocholate. Bile salts are bile acids compounded with a cation, usually sodium. Bile acids are steroid acids found predominantly in the bile of mammals and are commercially available. In one embodiment, the bile salts comprise cholate. In another embodiment, the bile salts comprises deoxycholate. In yet another embodiment, the bile salts comprise cholate and deoxycholate. In another embodiment, the bile salts consist of cholate and deoxycholate.

In certain embodiments, the concentration of NaCl is 1 mM to 1 M, 1 mM to 0.5 M, 1 mM to 0.1 M, 1 mM to 50 mM, 10 mM to 100 mM, 10 mM to 50 mM, 0.1 M to 1 M, 0.1 M to 0.5 M, or 0.5 M to 1 M. In certain embodiments, the concentration of the bile salts is 1 mM to 100 mM, 1 mM to 50 mM, 1 mM to 25 mM, 1 mM to 10 mM, 1 mM to 5 mM, 0.1 mM to 5 mM, 0.1 mM to 1 mM, or 0.1 mM to 0.5 mM bile salts.

These excipients are provided by way of example and it will be known to those of skill in the art that there will be other or different excipients that can provide the same chemical features as those listed herein.

In some embodiments, the cAMB of the present methods comprises the following components: amphotoricin B, a phospholipid, EDTA, water, vitamin E, calcium chloride, methylcellulose, methylparaben, proplyparaben, sodium hydroxide, dehydrated alcohol, monobasic potassium phosphate, potassium sorbate, acesulfame potassium and optionally flavoring.

In some embodiments, the cAMB of the present disclosure is formulated using the components and amounts described below in Table 2. In some embodiments, the cAMB of the present disclosure is formulated as a 27.5 mg/mL suspension, thus a 1000 mg dose is 36.4 mL.

TABLE 2
Formulation Composition of Cochleated Amphotericin B

| | Amount per Unit |
|---|---|
| Component | (mg/g) |
| Amphotericin B | 27.5 |
| Phospholipid, PS P50X^{†} | 137.5 |
| Purified Water | 770.15 |
| Edetate disodium (EDTA) | 0.68 |
| Vitamin E (dl-α-tocopherol) | 0.002 |
| Calcium chloride | 24.29 |
| Methylcellulose^{†} | 3.0 |
| Methylparaben^{†} | 1.8 |
| Propylparaben^{†} | 0.23 |
| Sodium hydroxide | 0.46 |
| Dehydrated Alcohol | 0.0095 |
| Monobasic potassium phosphate | 1.31 |
| Potassium sorbate | 2.0 |
| Acesulfame Potassium | 10 |
| N&A Flavor Enhancer | 10 |
| Art Strawberry Flavor | 10 |

^{†}U.S. FDA “Generally regarded as safe” (GRAS) designation as food additive or cosmetics.

### COMPOSITIONS

The present disclosure is also directed to a composition comprising encochleated amphotericin B (cAMB). In some embodiments, the encochleated cAMB comprises amphotoricin B, a phospholipid, EDTA, water, vitamin E, calcium chloride, methycellulose, methylparaben, proplyparaben, sodium hydroxide, dehydrated alcohol, monobasic potassium phosphate, potassium sorbate, acesulfame potassium and optionally flavoring. In some embodiments, the amounts of the foregoing components of the present encochleated cAMB are described in Table 2, above.

### EXAMPLES

### Example 1

### Safety and Tolerability (Phase I)

Materials and Methods

We conducted a Phase 1 safety and tolerability trial for cAMB in HIV-positive Ugandans from October 2019 to January 2020 in Kampala, Uganda. The trial was designed to occur in two sequential phases, with 27 participants enrolled in Phase 1A and 9 in Phase 1B.

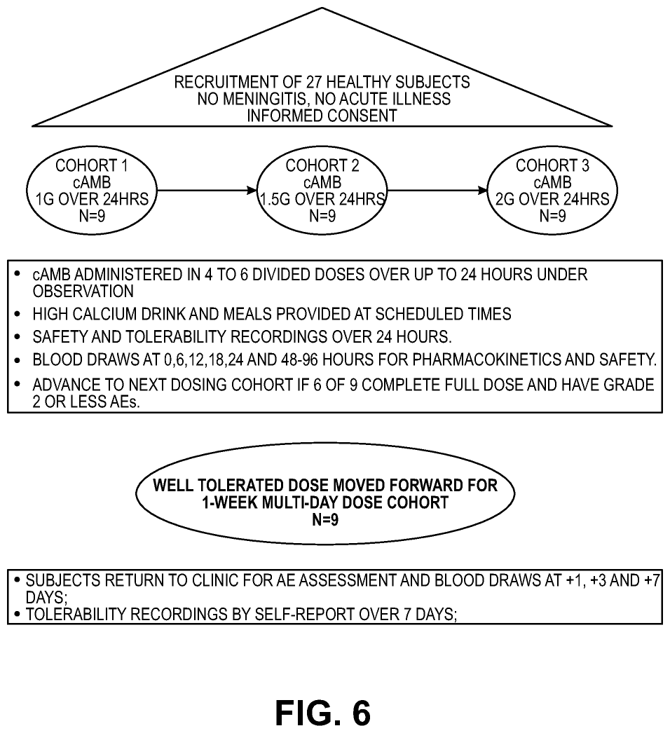

The cAMB was administered at 1.0 g, 1.5 g, and 2.0 g per day in 4-6 divided doses according to FIG. 6, with 9 participants in each dose group. At least 6 of 9 participants had to tolerate the dose before proceeding to the next dosing cohort. Dose-limiting intolerability was defined as experiencing a Grade 3 or higher AE, vomiting within 30 minutes after taking a dose, or permanently discontinue taking the medication due to an AE or toxicity. The participants in Phase 1B received the cAMB dose that was 100% tolerated in Phase 1A for 7 days to verify safety and tolerability with continuous dosing. Participants in Phase 1A and 1B had the same inclusion and exclusion criteria.

Participants were recruited from a prior cryptococcal meningitis trial cohort (ASTRO-CM; ClinicalTrials.gov: NCT01802385) (Rhein et al., 2019. Lancet Infect. Dis. 19:843-851). All recruited subjects had a history of resolved HIV-associated cryptococcal meningitis and had received IV AMB previously. Inclusion requirements were age >18 years and a calculated creatinine clearance >70 mL/min/1.73 m² (measured within 3 months). Participants were excluded if they exhibited a current illness, had a known untreated significant health problem, or had received amphotericin B therapy in the past 90 days. Women who were pregnant or breastfeeding were excluded. rahics, vital signs and HIV characteristics are summarized in Table 3.

Trial endpoints included: 1) Proportion of daily dose received and tolerated without vomiting within 30 minutes; 2) Cumulative score of nausea, vomiting, diarrhea by Common Terminology Criteria AE (CTCAE) grade scoring criteria (U.S. Department of Health and Human Services NIoH, National Cancer Institute, 2017, Common Terminology Criteria for Adverse Events (CTCAE) v 5.0); 3) Incidence of adverse events (AEs) by National Institute of Allergy and Infectious Diseases (NIAID) Division of AIDS (DAIDS) toxicity scale (version 2017) for Grade 1-5 or serious AEs (SAE) (U.S. Department of Health and Human Services NIoH, National Institute of Allergy and Infectious Diseases, Division of AIDS, 2017, Division of AIDS (DAIDS) Table for Grading the Severity of Adult and Pediatric Adverse Events, Corrected Version 2.1.); 4) Participant subjective impression(s) of cAMB by visual analog and other scales. All clinical and laboratory AEs were followed up until resolution.

We collected plasma for pharmacokinetic analyses at 0, 6, 12, 24, and 48 hours in Phase 1A. We monitored complete blood count, chemistries, kidney and liver function tests at 0, 24, and 48 hours, with follow up thereafter of any abnormal results. For Phase 1B, the same laboratory monitoring occurred at +1 day, +3 days, and +7 days for safety assessment and pharmacokinetic analyses. All laboratory measurements were performed at the IDI core laboratory, which is a College of American Pathologists (CAP)-certified laboratory and participates in external quality assurance for all laboratory assays.

Statistical analyses were primarily descriptive. The median and interquartile ranges or the number and proportions were reported where appropriate. Area under the curve (AUC) calculations were made using a linear trapezoidal method with statistical comparison by ANOVA. A visual analog scale was used to assess participants' subjective experience with various aspects of the trial. The number of clinical and laboratory adverse events (AE) were summarized by the Division of AIDS (DAIDS) Toxicity Table, version 2017.

The Mulago Hospital Research and Ethics Committee, the Ugandan National Council of Science and Technology, the Uganda National Drug Authority, and the University of Minnesota IRB approved the study. All participants provided informed consent in their preferred language. An internal review was performed after each dosing cohort to assure tolerability. A data safety monitoring board (DSMB) reviewed all safety data after completion of Phase 1 data collection.

Results

In the initial single ascending dose trial, a total of 35 HIV-positive cryptococcal survivors were screened. We enrolled 9 participants into each of the three sequential dosing cohorts: 1.0 g, 1.5 g, and 2.0 g per day. All participants were survivors of prior cryptococcal meningitis (median time since prior diagnosis was 46 months) and had previously received about 14 days IV AMB. The median age was 36 years (IQR, 30-44) with 41% (11/27) being women. All participants were currently receiving antiretroviral therapy (ART) of which 15 (42%) were taking atazanavir, and 37% (10/27) were actively taking fluconazole 200 mg prophylaxis.

Meeting tolerability criteria required participants to take the full daily dose without vomiting within 30 minutes after taking any dose, report AEs of grade 2 or less, and not discontinue the cAMB due to an AE or participant choice. For the 1.0 g and 1.5 g cohorts, all 18 participants met full tolerability criteria. In the 2.0 g cohort, all 9 participants completed dosing, but one participant experienced a grade 3 laboratory AE of transient thrombocytopenia at 48 hours of unclear etiology. Therefore, only 89% (8/9) participants met full tolerability criteria at 2.0 g dosing.

Of the 27 participants in Phase 1A, 8 (30%) experienced at least one clinical AE. A total of 31 unique clinical AEs were reported, with 24 (77%) as grade 1 and 7 (23%) as grade 2. Increasing numbers of AEs correlated with increasing cAMB dose, with 65% (20/31) recorded in the 2.0 g cohort. No grade 3, grade 4, or serious AEs occurred. The most common clinical AEs were abdominal pain (n=5), nausea (n=5), and dizziness (n=4). Of the same 27 participants, 15 (56%) experienced at least one laboratory AE. A total of 24 laboratory AEs were reported, with 14 (58%) as grade 1 and 9 (38%) as grade 2. There was one grade 3 adverse event where a participant in the 2.0 g cohort had platelet counts of 163,000 and 160,000 cells/μL at baseline and 24 hours, respectively; then dropped to 31,000 cells/μL at 48 hours. No platelet clumping was reported. The platelet count was repeated several times to ensure AE resolution; the count rebounded to 172,000 cells/μL by two weeks. The participant had no clinical deterioration or evidence of bleeding at the time of the documented thrombocytopenia. No grade 4 nor serious laboratory AEs occurred. The most common laboratory AEs were total bilirubin elevation (n=6); mild isolated AST elevation (n=5), and mild hypernatremia (n=5) which met DAIDS criteria for Grade 1 AE (146-149 mEq/L) but was within the normal range of the local CAP-certified laboratory for the population (138-150 mEq/L). Of AST elevations, only one was accompanied by an ALT elevation in a subject with chronic hepatitis B. Adverse events are summarized in Table 4.

A dose of 1.5 g was selected for Phase 1B as the “100% tolerated” Phase 1A dose. The demographics of the 9 Phase 1B participants were similar to those of Phase 1A. Eight of the 9 participants completed full dosing through 7 days, with one participant unintentionally missing one day of dosing. Three participants experienced a total of five Grade 1 clinical AEs, none being serious. Three participants also experienced a total of six Grade 1 laboratory AEs, again none meeting criteria for a serious AE. Overall, the participants from Phase 1B took cAMB successfully as outpatients and displayed minimal evidence of intolerance or toxicity with 7 days of continuous therapy. Adverse events are summarized in Table 4.

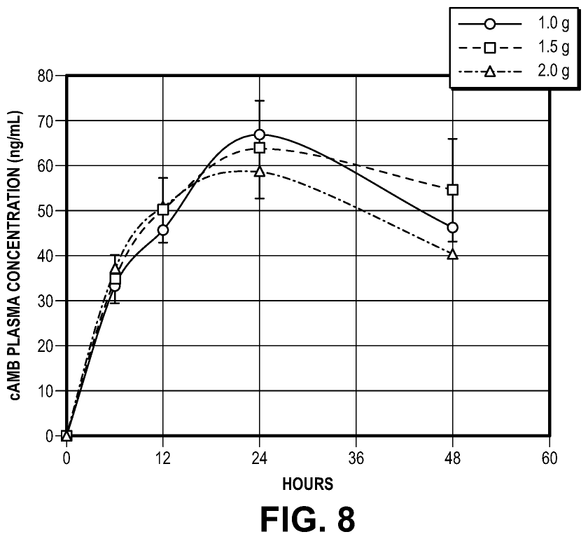

Plasma concentrations of cAMB were measured at 0, 6, 12, 24, and 48 hours from time of cAMB initiation and area under the curve (AUC) was calculated. FIG. 8, which depicts the mean cAMB plasma concentrations at various time points, shows that the plasma AUC₀₋ₗₐₛₜ did not significantly differ across the three dosing cohorts averaging 2350 ng*h/mL (P=). The error bars in FIG. 8 represent the 95% confidence interval for the 1.5 g cohort. P-value calculated by one-way ANOVA.

The half-life of cAMB was 53 hours, and the mean peak Cₘₐₓ was 63.0 ng/mL (±16.2) at 24 hours (averaged across the cohorts). In pharmacokinetic analyses of the phase 1B cohort, plasma levels at 24 hours (74.8 ng/mL) were 77% of day 7 levels of 97.7 ng/mL. At 72 hours, plasma levels of 91.1 ng/mL were 93% of day 7 levels.

Using a qualitative scale, we assessed participant impressions of their cAMB experience versus their prior IV AMB experience in the Phase 1A cohort. In the pre-participation survey, 40% (10/25) indicated that they would be willing to receive IV AMB again for their cryptococcal meningitis versus an alternative therapy. After completing study follow-up, 96% (26/27) of participants indicated they would prefer cAMB to IV AMB for cryptococcal meningitis treatment. Overall 93% (25/27) thought the multiple doses of cAMB was very convenient for therapy compared to 65% (15/23) for IV AMB of respondents. One participant could not recall their initial IV AMB therapy. Nausea severity scoring was rated as “none” for 8 of 9 participants in the 1 g and 1.5 g cohorts, and 7 of 9 for the 2.0 g cohort. In comparison, 25% (6/24) recalled severe nausea with IV AMB.

TABLE 3
Baseline characteristics

| | Phase IA | | | | | | | |
|---|---|---|---|---|---|---|---|---|
| | Cohort 1 | | Cohort 2 | | Cohort 3 | | Phase IB | |
| | (1.0 g) | | (1.5 g) | | (2.0 g) | | (1.5 g) | |
| No. participants enrolled | 9 | | 9 | | 9 | | 9 | |
| | N (%) or | | N (%) or | | N (%) or | | N (%) or | |
| Demographics | Median [IQR] | | Median [IQR] | | Median [IQR] | | Median [IQR] | |
| Age | 33.0 | [30.0, 44.0] | 39.0 | [30.0, 43.0] | 42.0 | [35.0, 44.0] | 34.0 | [31.0, 40.0] |
| Female | 4 | (44.4%) | 2 | (22.2%) | 5 | (55.6%) | 5 | (55.6%) |
| Height (cm) | 165.8 | [158.2, 168.5] | 165.7 | [163.3, 167.2] | 166.0 | [153.2, 168.8] | 164.2 | [156.4, 165.8] |
| Weight (kg) | 63.5 | [60.5, 68.5] | 57.5 | [55.5, 68.0] | 75.0 | [65.5, 78.0] | 69.5 | [61.5, 79.8] |
| Vital Signs | Median [IQR] | | Median [IQR] | | Median [IQR] | | Median [IQR] | |
| Sytolic BP | 108.0 | [104.0, 112.0] | 124.0 | [120.0, 132.0] | 112.0 | [107.0, 125.0] | 127.0 | [113.0, 136.0] |
| Diastolic BP | 70.0 | [60.0, 72.0] | 79.0 | [73.0, 88.0] | 73.0 | [68.0, 83.0] | 78.0 | [69.0, 82.0] |
| Pulse | 69.0 | [64.0, 74.0] | 70.0 | [69.0, 79.0] | 65.0 | [60.0, 78.0] | 74.0 | [66.0, 83.0] |
| | N (%) or | | N (%) or | | N (%) or | | N (%) or | |
| HIV History | Median [IQR] | | Median [IQR] | | Median [IQR] | | Median [IQR] | |
| HIV positive | 9 | (100.0%) | 9 | (100.0%) | 9 | (100.0%) | 9 | (100.0%) |
| Currently on ART¹ | 9 | (100.0%) | 9 | (100.0%) | 9 | (100.0%) | 9 | (100.0%) |
| Months on ART | 41.9 | [10.7, 43.2] | 28.6 | [16.1, 52.4] | 42.0 | [19.7, 47.0] | 41.4 | [18.6, 44.6] |
| CM History | N (%) | | N (%) | | N (%) | | N (%) | |
| Previous blood CrAg screen | 9 | (100.0%) | 9 | (100.0%) | 9 | (100.0%) | 9 | (100.0%) |
| Previous history of CM | 9 | (100.0%) | 9 | (100.0%) | 9 | (100.0%) | 9 | (100.0%) |
| | N (%) or | | N (%) or | | N (%) or | | N (%) or | |
| HIV History | Median [IQR] | | Median [IQR] | | Median [IQR] | | Median [IQR] | |
| Months since last diagnosis | 45.3 | [44.3, 46.2] | 48.1 | [41.4, 51.9] | 49.1 | [44.7, 51.1] | 48.5 | [47.8, 50.6] |
| Prior IV amphotericin | 9 | (100.0%) | 9 | (100.0%) | 9 | (100.0%) | 9 | (100.0%) |

TABLE 4
Adverse Event Summary

| | Phase IA | | | |
|---|---|---|---|---|
| | Cohort 1 | Cohort 2 | Cohort 3 | Phase IB |
| | (1.0 g) | (1.5 g) | (2.0 g) | (1.5 g) |
| No. participants | 9 | 9 | 9 | 9 |
| No. participants with | 0 | 0 | 0 | 0 |
| serious adverse event | | | | |
| No. participants with | 0 | 0 | 0 | 0 |
| unexpected event | | | | |
| No. clinical events (%) | 4 | 7 | 20 | 5 |
| Grade 1 | 4 (100) | 3 (43) | 17 (85) | 5 (100) |
| Grade 2 | 0 | 4 (57) | 3 (15) | 0 |
| Grade 3 | 0 | 0 | 0 | 0 |
| Grade 4 | 0 | 0 | 0 | 0 |
| No. laboratory events | 8 | 5 | 11 | 6 |
| (%) | | | | |
| Grade 1 | 2 (25) | 4 (80) | 8 (73) | 6 (100) |
| Grade 2 | 6 (75) | 1 (20) | 2 (18) | 0 |
| Grade 3 | 0 | 0 | 1 (9) | 0 |
| Grade 4 | 0 | 0 | 0 | 0 |

### Example 2

### Efficacy (Phase II)

The efficacy phase (Phase II) of the trial will be a prospective randomized study to evaluate the safety, tolerability, and microbiologic efficacy of oral encholeated amphotericin (cAMB) compared to IV amphotericin (AMB) for induction therapy. Participants enrolled in the experimental arm will receive oral cAMB +5-Flucocytosine (5-FC) in four stages of duration for induction therapy, using the maximal tolerated dose of cAMB found in the Phase I trial, i.e., the dose wherein at least 66% (6 of 9) of participants receiving the full daily dose have Grade <2 GI toxicity and Grade <2 lab AE through up to 48-96 hours. The experimental arm will receive cAMB through 6 weeks. Participants randomized to the control arm will receive the 2018 WHO recommended standard of care.² There will be a scheduled enrollment pause after each stage. ² World Health Organization Guidelines for the diagnosis, prevention and management of cryptococcal disease in HIV-infected adults, adolescents and children. 2018; accessible via the world wide web at who.int/hiv/pub/guidelines/cryptococcal-disease/en/. Accessed Apr. 1, 2018, which is herein incorporated in its entirety.

Briefly, the 2018 WHO recommended standard of care is as follows. For adults, adolescents and children, a short-course (one-week) induction regimen with amphotericin B deoxycholate (1.0 mg/kg/day) and flucytosine (100 mg/kg/day, divided into four doses per day), followed by 1 week of fluconazole (1200 mg/day for adults, 12 mg/kg/day for children and adolescents, up to a maximum dose of 800 mg daily), is the preferred option for treating cryptococcal meningitis among people living with HIV (strong recommendation, moderate certainty evidence for adults, low-certainty evidence for children and adolescents). Alternative induction regimens depending on drug availability are (1) two weeks of fluconazole (1200 mg daily for adults, 12 mg/kg/day for children and adolescents)+flucytosine (100 mg/kg/day, divided into four doses per day) (strong recommendation, moderate-certainty evidence); or (2) Two weeks of amphotericin B deoxycholate (1.0 mg/kg/day)+fluconazole (1200 mg daily for adults, 12 mg/kg/day for children and adolescents up to a maximum of 800 mg daily) (strong recommendation, moderate-certainty evidence).

Fluconazole (800 mg daily for adults, 6-12 mg/kg/day for children and adolescents up to a maximum of 800 mg daily) is recommended for the consolidation phase (for eight weeks following the induction phase) (strong recommendation, low-certainty evidence). Fluconazole (200 mg daily for adults, 6 mg/kg/day for adolescents and children) is recommended for the maintenance phase (strong recommendation, high-certainty evidence).

Participants

Participants for the efficacy phase of the study include individuals with cryptococcal meningitis, including HIV patients, who are diagnosed from a CSF fungal culture positive for Cryptococcus species and/or who are CSF cryptococcal antigen (CRAG) positive, and are willing to receive protocol-specified lumbar punctures. Subjects with a previous history of prior, known cryptococcal meningitis will be included in the trial. The rationale for including this patient population is that there is a strong association between disease relapse and fluconazole resistance, and we therefore believe that the addition of cAMB could be highly beneficial in this patient population.

This phase of the study excludes those individuals who are less than 18 years of age, have a Glasgow coma scale of <15, have received three or more doses of IV amphotericin B therapy within the last 30 days, are unable to take enteral medicine, are pregnant or breastfeeding, cannot or are unlikely to attend regular clinic visits, receive chemotherapy or corticosteroids, are suspected of having paradoxical immune reconstitution inflammatory syndrome (IRIS) or have recently initiated HIV therapy or antiretroviral therapy (ART) class switch (within 2 weeks).

Treatment Regimens

Stage 1

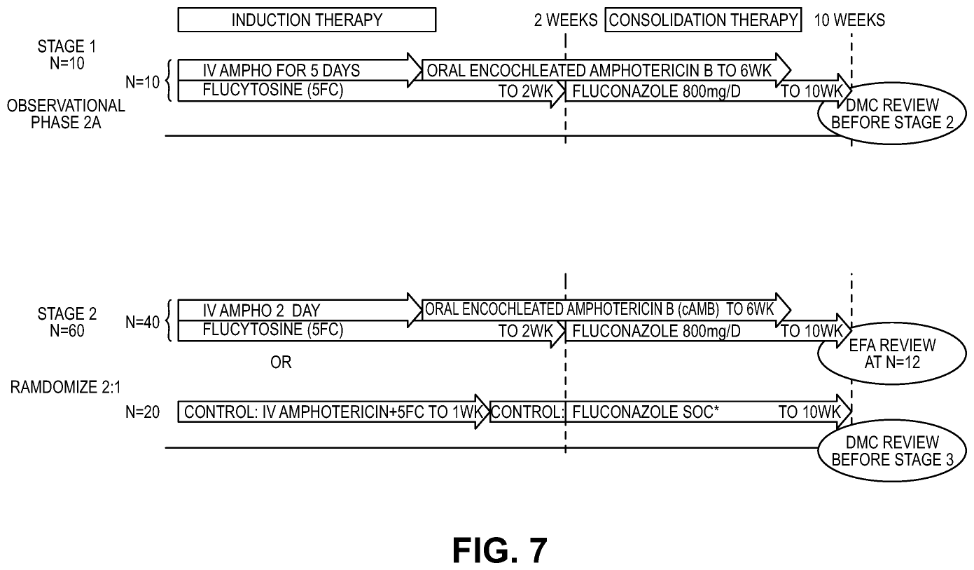

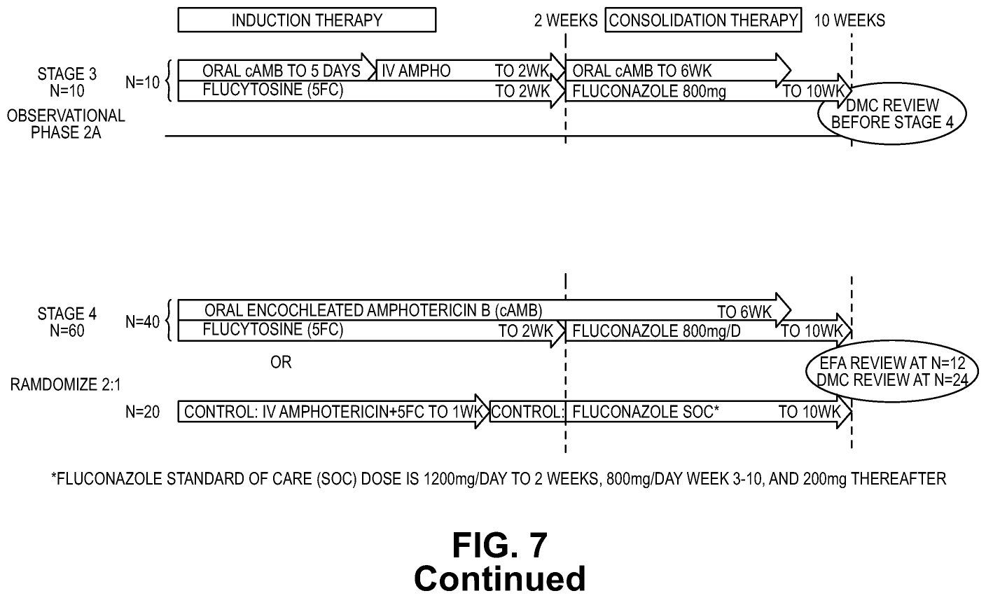

As depicted in FIG. 7, the efficacy phase of the study includes four stages. The first stage of the trial includes a 10 person non-randomized observational Phase 2A study to assess for safety and tolerability of the cAMB study drug in a cryptococcal meningitis population. The Stage 1 experimental arm receives induction therapy comprising 1a) IV Amphotericin B deoxycholate 1 mg/kg/day from day 1 to day 5; oral cAMB from day 5 to day 14 2a) oral 5-FC, 100 mg/kg/day, from day 1 to day 14. The dosage of orally administered cAMB during the induction treatment phase is the divided dose amount found to be tolerated by 6 of the 9 Phase I trial participants with Grade 2 or less adverse advents.

The induction treatment phase of the Stage 1 study is followed by a consolidation treatment phase, which comprises 1b) oral cAMB from day 15 to week 6; 2b) oral fluconazole, 800 mg/day, from day 15 to week 10 and 3b) fluconazole, 200 mg/day thereafter (maintenance therapy). The dosage of orally administered cAMB during the consolidation treatment phase is the divided dose amount found to be tolerated by 9 of the 9 Phase I trial participants with Grade 2 or less adverse advents. Upon confirmation of the safety of the Stage 1 dosage scheme, the study will proceed to Stage 2.

Stage 2

In Stage 2, participants will be randomized 2:1 to experimental arm (n=40) versus control arm (n=20). This stage is testing the efficacy and safety of a shortened duration of IV AMB therapy in comparison to the controls receiving standard of care IV AMB.

The Stage 2 experimental arm receives induction therapy comprising 1a) IV AMB, 1 mg/kg/day, from day 1 to day 2; oral cAMB from day 2 to day 14 2a) oral 5-FC, 100 mg/kg/day, from day 1 to day 14. The dosage of orally administered cAMB during the induction phase is the divided dose amount found to be tolerated by 6 of the 9 Phase I trial participants with Grade 2 or less adverse advents.

The induction therapy of the Stage 2 study is followed by a consolidation treatment phase, which comprises 1b) oral cAMB from day 15 to week 6; 2b) oral fluconazole 800 mg/day from day 15 to week 10 and 3b) fluconazole at 200 mg/day thereafter (maintenance therapy). The dosage of orally administered cAMB during the consolidation treatment phase is the divided dose amount found to be tolerated by 9 of the 9 Phase I trial participants with Grade 2 or less adverse advents.

There will be an early safety review focused on the EFA CSF clearance rate after the 12^{th} overall subject (20% of the stage) accrues >2 weeks of data. This early safety check will focus on CSF EFA only. If there are any concerns over the CSF clearance rate in the experimental arm, then more frequent EFA safety reviews may be undertaken. The CSF clearance EFA, safety, survival, and tolerability data after the 60^{th} subject accrues >4 weeks of follow up data is also evaluated.

Participants randomized to the control arm receive (as per 2018 WHO Guidelines)³ induction therapy comprising 1a) IV AMB at 1 mg/kg/day from day 1 to day 7; 2a) oral 5-FC at 100 mg/kg/day from day 1 to day 7. The induction therapy is followed by a consolidation treatment phase comprising oral fluconazole at 800 mg/day from day 15 to week 10, after which the participants receive maintenance therapy, i.e., oral fluconazole at 200 mg/day. ³ Id.

The 2018 WHO recommended regimen is based on a Phase III randomized clinical trial demonstrating that: 1) 1 week of IV AMB had equivalent survival as 2 weeks of IV AMB; 2) 1 week of IV AMB combination therapy was less toxic than 2 weeks of IV AMB; and 3) adjunctive 5-FC had superior survival to adjunctive fluconazole.

Stage 3

Stage 3 is an observational, Phase 2A safety cohort to assess for the safety of starting initial induction therapy with oral cAMB and 5-FC. Ten participants will receive 5 days of oral cAMB before switching to scheduled IV AMB therapy. The primary objective of this stage is to assess the initial CSF clearance rate (i.e. EFA) in the absence of IV AMB loading doses, so as to make a determination on the safety to proceed to Stage 4.

The Stage 3 experimental arm receives induction therapy comprising 1a) oral cAMB from day 1 to day 5 2a) IV AMB at 1 mg/kg/day from day 6 to day 14 3a) oral 5-FC at 100 mg/kg/day from day 1 to day 14. The dosage of orally administered cAMB during the induction phase is the divided dose amount found to be tolerated by 6 of the 9 Phase I trial participants with Grade 2 or less adverse advents.

The induction therapy of the Stage 3 study is followed by a consolidation treatment stage, which comprises 1b) oral cAMB from day 15 to week 6; 2b) oral fluconazole at 800 mg/day from Day 15 to Week 10 and 3b) fluconazole at 200 mg/day thereafter (maintenance therapy). The dosage of orally administered cAMB during the consolidation treatment phase is the divided dose amount found to be tolerated by 9 of the 9 Phase I trial participants with Grade 2 or less adverse advents. Upon confirmation of the safety of the Stage 1 dosage scheme, the study will proceed to Stage 4. There is no control arm for Stage 3.

The CSF clearance EFA, safety, survival, and tolerability data after the 10^{th} subject accrues ≥4 weeks of follow up data is also evaluated. Controls from Stage 2 will be available for non-statistical comparison regarding expected outcomes.

Stage 4

In Stage 4, participants will be randomized 2:1 to experimental arm (n=40) versus control arm (n=20). The dosage schedule for the control arm is the same as described above for Stage 2. This stage tests whether oral cAMB can be used without IV AMB.

The Stage 4 experimental arm receives induction therapy comprising 1a) oral cAMB from day 1 to day 14 and 2a) oral 5-FC at 100 mg/kg/day from day 1 to day 14. The dosage of orally administered cAMB during the induction phase is the divided dose amount found to be tolerated by 6 of the 9 Phase I trial participants with Grade 2 or less adverse advents.

The induction therapy of the Stage 4 study is followed by a consolidation treatment stage, which comprises 1b) oral cAMB from day 15 to week 6; 2b) oral fluconazole at 800 mg/day from day 15 to week 10 and 3b) fluconazole at 200 mg/day thereafter (maintenance therapy). The dosage of orally administered cAMB during the consolidation treatment phase is the divided dose amount found to be tolerated by 9 of the 9 Phase I trial participants with Grade 2 or less adverse advents.

There will be an early safety review focused only on the EFA CSF clearance after the 12^{th} overall subject (20% of the stage) accrues >2 weeks of data. This early safety check will focus on EFA CSF only. If there is any concern over the CSF clearance rate in the experimental arm, then more frequent reviews may be undertaken. The CSF clearance EFA, safety and tolerability data after the 24^{th} subject (40% of the stage) accrues ≥4 weeks of follow up data is also evaluated. Enrollment will continue unless there are investigator concerns.

Participants in all four stages will receive lumbar punctures (LPs) at diagnosis, day 3, day 5-7, day 10-14, and additionally as required for control of intracranial pressure and documentation of CSF sterilization. Therapeutic LPs conducted during the first week have a ˜70% relative survival benefit.

1. A method of treating or preventing a fungal infection in a subject comprising a Cryptococcus spp., which method comprises an induction treatment phase and a consolidation treatment phase,
wherein the induction treatment phase comprises:
administering encochleated amphotericin B (cAMB) and 5-Flucytosine or an azole to the subject, wherein the cAMB and the 5-Flucytosine or the azole are mucosally administered.

2. The method of claim 1, wherein the induction treatment phase comprises administering the 5-Flucytosine to the subject.

3. The method of claim 1, wherein the muscosal administration is oral administration.

4. The method of claim 3, wherein the oral administration of cAMB to the subject during the induction phase comprises orally administrating a divided dosage of cAMB.

5. The method of claim 1, wherein a cumulative daily dosage of the cAMB administered to the subject during the induction treatment phase is at least 1 gram per day, 1.5 grams per day, or 2 grams per day.

6. (canceled)

7. (canceled)

8. The method of claim 1, wherein the induction treatment phase is 14 days and wherein the cAMB is administered on day 1 to day 5, day 5 to day 14 or day 3 to day 14 of the induction treatment phase.

9. The method of claim 1, wherein the induction treatment phase is 14 days and wherein the cAMB is administered to the subject on day 1 to day 14.

10. The method of claim 1, wherein the induction treatment phase further comprises intravenously administering unencochleated amphotericin B to the subject.

11. The method of claim 10, wherein the induction treatment phase is 14 days and wherein the intravenously administered unencochleated amphotericin B is intravenously administered on day 1 to day 2, day 1 to day 5, or day 6 to day 14 of the 14 day induction treatment phase.

12. The method of claim 1, wherein the 5-Flucytosine is administered to the subject in the induction treatment phase for at least two weeks.

13. The method of claim 1, wherein the induction treatment phase comprises administering 100 mg/kg/day of 5-Flucytosine to the subject.

14. The method of claim 1, wherein the consolidation treatment phase comprises orally administering cAMB and an azole to the subject.

15. The method of claim 1, wherein the azole is fluconazole.

16. The method of claim 1, wherein the consolidation treatment phase is ten weeks and wherein the cAMB is orally administered to the subject during the consolidation treatment phase for four weeks of the ten week consolidation treatment phase.

17. The method of claim 1, wherein a divided dosage of cAMB is orally administered to the subject during the consolidation treatment phase.

18. The method of claim 1, wherein fluconazole is orally administered to the subject in the consolidation treatment phase for at least ten weeks.

19. The method of claim 1, wherein fluconazole is orally administered to the subject in the consolidation treatment phase and wherein a dosage of the fluconazole in the consolidation treatment phase is about 800 milligrams/day.

20. The method of claim 1, wherein cAMB is orally administered during the consolidation treatment phase and wherein a dosage of the cAMB in the consolidation treatment phrase is at least 1.5 grams per day.

21. The method of claim 1, wherein the subject is suffering from cryptococcus meningitis.

22. The method of claim 1, wherein the method further comprises a maintenance phase, the maintenance phase comprising orally administering fluconazole to the subject.

23. The method of claim 22, wherein the fluconazole is administered at a dosage of about 200 mg/day during the maintenance phase.

24. The method of claim 1, wherein the Cryptococcus spp. is Cryptococcus neoformans or Cryptococcus gattii.

25. (canceled)

26. The method of claim 1, wherein the subject is a human.

27. The method of claim 1, wherein the subject has HIV/AIDS, lymphoma, cirrhosis of the liver or has received an organ transplant.

28. The method of claim 1, wherein the subject is an HIV-infected subject.

29. The method of claim 1, wherein the Cryptococcus spp. infecting the subject exhibits reduced sensitivity or resistance to fluconazole.

30. A composition comprising encochleated amphotericin B, wherein the encochleated amphotericin B comprises amphotericin B, a phospholipid, EDTA, water, vitamin E, calcium chloride, methylcellulose, methylparaben, proplyparaben, sodium hydroxide, dehydrated alcohol, monobasic potassium phosphate, potassium sorbate, acesulfame potassium and optionally flavoring.
